(19) United States
(12) Patent Application Publication
(10) Pub. No.: US 2013/0310439 A1
(43) Pub. Date: Nov. 21, 2013

(54) METHOD OF REDUCING PROTEINS MISFOLDING AND/OR AGGREGATION

(71) Applicants: Universidade De Lisboa, Lisboa (PT); Northwestern University, Evanston, IL (US)

(72) Inventors: Richard I. Morimoto, Evanston, IL (US); Maria Catarina Telo Baptista Lima Da Silva, Evanston, IL (US)

(73) Assignees: Universidade De Lisboa; Northwestern University

(21) Appl. No.: 13/669,070

(22) Filed: Nov. 5, 2012

Related U.S. Application Data
(63) Continuation of application No. PCT/US11/35317, filed on May 5, 2011.
(60) Provisional application No. 61/331,678, filed on May 5, 2010.

(51) Int. Cl. A61K 31/7088

(52) U.S. Cl. CPC A61K 31/7088
USPC 514/44 A; 435/29

(57) ABSTRACT

The present invention is directed to methods of reducing protein misfolding and/or aggregation in a subject and to method of treating a condition mediated by a dysfunction in protein homeostasis comprising modulating cholinergic signaling activity.

### RELATED APPLICATIONS

This application is a continuation of International Application No. PCT/US11/35317, which designated the United States and was filed on May 5, 2011, published in English, which claims the benefit of U.S. Provisional Application No. 61/331,678, filed on May 5, 2010. The entire teachings of the above application(s) are incorporated herein by reference.

### GOVERNMENT SUPPORT

This invention was made with government support under Grant No.'s R37GM038109 and R01 AG026647 awarded by the National Institutes of Health. The government has certain rights in the invention.

### BACKGROUND OF THE INVENTION

Cells normally maintain a balance between protein synthesis, folding, trafficking, aggregation, and degradation, referred to as protein homeostasis, utilizing sensors and networks of pathways [Sitia et al., Nature 426: 891-894, 2003; Ron et al., Nat Rev Mol Cell Biol 8: 519-529, 2007]. Protein homeostasis, or proteostasis, is maintained by controlling the conformation, binding interactions, location and concentration of individual proteins making up the proteome. Disruption of proteostasis results in the accumulation of intermediate normative conformations that form oligomeric and/or aggregated protein species that ultimately cause cell injury over time [Garcia et al. (2007). Genes and Development 21: 3006-16]. Human gain of function diseases are often the result of a disruption in protein homeostasis leading protein aggregation [Balch et al. (2008), Science 319: 916-919]. Human loss of function diseases are also the result of a disruption of normal protein homeostasis, typically caused by a mutation in a given protein that compromises its cellular folding, leading to efficient degradation [Cohen et al., Nature 426: 905-909, 2003].

Protein misfolding and/or aggregation have been implicated in a diverse range of diseases including for example, neurodegenerative diseases, metabolic diseases, inflammatory diseases and cardiovascular diseases. There remains a need in the art for therapeutic approaches to treat conditions associated with proteostasis dysfunction.

### SUMMARY OF THE INVENTION

The present invention is based on the surprising discovery that increased cholinergic signaling at the synapse activates the stress response in the post-synaptic cell, resulting in amelioration of protein misfolding and/or aggregation in post-synaptic cells. For example, Example 1 shows that inhibition of GEI-11, a negative regulator of the acetylcholine receptor (AchR), suppressed protein aggregation in Caenorhabditis (C.) elegans.

The present invention is directed to methods of reducing protein misfolding and/or aggregation in a subject and to methods of treating a condition mediated by a dysfunction in protein homeostasis comprising modulating cholinergic signaling activity.

In one aspect, the invention is a method of reducing cholinergic hypostimulation-associated protein misfolding and/or aggregation in a subject comprising administering to said subject an effective amount of an agent that increases cholinergic signaling or an agent that decreases GABAergic signaling. In one embodiment, the agent that increases cholinergic signaling is an inhibitor of GEI-11 or a homolog thereof.

In another aspect of the invention, the method is directed to treating a condition mediated by a dysfunction in protein homeostasis in a patient in need thereof comprising administering to said patient an effective amount of an agent that increases cholinergic signaling or an agent that decreases GABAergic signaling. In one aspect, the condition is a gain of function disorder. In another aspect, the condition is a loss of function disorder. In yet another embodiment, the condition is a muscle wasting condition.

In another aspect, the invention is a method of reducing cholinergic hyperstimulation associated protein misfolding and/or aggregation in a subject comprising administering to said subject an effective amount of an agent that decreases cholinergic signaling or an agent that increases GABAergic signaling.

The invention also encompasses a method of reducing protein misfolding and/or aggregation in a post-synaptic muscle cell comprising decreasing cholinergic signaling at the neuromuscular junction.

In another aspect, the invention is directed to a method of screening for an agent that reduces protein misfolding, aggregation or both in a post-synaptic cell comprising screening a candidate agent for cholinergic agonist activity.

### BRIEF DESCRIPTION OF THE DRAWINGS

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

FIG. 1A: One day old (L1s) Q35 animals were treated with 25 μM levamisole or 1 mM nicotine. The white boxes indicate magnified areas shown below. The photographs are from 4 days old animals. Scale bars: controls=200 μm, nicotine, levamisole, lower right panels=50 μm; and others=100 μm.

FIG. 1B are bar graphs showing the number of aggregates in Q35 animals treated with nicotine, levamisole or control animals as a function of age (days).

FIG. 2 is a schematic representation of neuronal signaling at the neuromuscular junction and how the compounds tested affect GABA or ACh signaling in muscle cells. ACh is acetylcholine. AChR is acetylcholine receptor. GABA is γ-aminobutyric acid. GABAR is GABA receptor.

FIG. 3A shows fluorescent microscopy images of Q35 animals fed gei-11 RNAi-containing bacteria or control bacteria.

FIG. 3B is a bar graph showing % phenotype in multiple temperature-sensitive (TS) mutant strains (described below) for the respective TS phenotypes, grown on gei-11 RNAi plates at a sensitized temperature of 23° C. and controls (L1 nematodes were grown on control bacteria (L4440 vector RNAi) at either 15° C., 23° C. or at 15° C. until L3-L4 stage and then transferred to 25° C.).

FIG. 4 is a schematic of the role of GEI-11 in regulating cholinergic signaling at the neuromuscular junction.

FIG. 5 are graphs showing relative mRNA levels of the acetycholine receptor subunits (top graph) and chaperones (bottom graph) in N2 animals fed L4440 bacteria (empty vector/control), N2 animals fed gei-11 RNAi-containing bacteria and an ACh receptor mutant strain (for subunit UNC-29) fed gei-11 RNAi-containing bacteria; for each of the indicated gene primers.

FIG. 6 is a schematic depicting the imbalance in cholinergic signaling caused by gei-11 knockdown and consequent activation of the heat shock response and restoration of proteostasis.

### DETAILED DESCRIPTION OF THE INVENTION

A description of preferred embodiments of the invention follows.

The words “a” or “an” are meant to encompass one or more, unless otherwise specified.

The term “agent” is used herein to denote a chemical compound, a mixture of chemical compounds, a biological macromolecule (including, for example, a nucleic acid, an antibody, a protein or portion thereof, e.g., a peptide), or an extract made from biological materials such as bacteria, plants, fungi, or animal (particularly mammalian) cells or tissues.

As used herein, the terms “inhibiting,” “decreasing” or “reducing” encompasses causing a net decrease by either direct or indirect means. The terms “increasing” or “enhancing” means to cause a net gain by either direct or indirect means.

The present invention is based on the discovery that changes in cholinergic signaling at the neuromuscular junction can affect proteostasis in the post-synaptic cell. It was previously described that neuronal hyperexcitation, caused either by cholinergic hyperstimulation or defective GABAergic signaling, resulted in premature aggregation of mutant polyglutamine expansion (polyQ) proteins in the body wall muscle cells in a Caenorhabditis (C.) elegans (Garcia et al. (2007). Genes and Development 21: 3006-16; the contents of which are expressly incorporated by reference herein). As described below, it has been surprisingly found that inhibition of GEI-11, a negative regulator of UNC-29 and the acetylcholine receptor (AchR), suppresses protein misfolding and aggregation in C. elegans. Therefore, both cholinergic hyperstimulation and cholinergic hypostimulation increase the appearance of misfolded and/or aggregated proteins. Proteostasis can be maintained and/or can be restored by achieving a requisite balance between cholinergic signaling and GABAergic signaling. There is a threshold level of cholinergic signaling, above and below which, the appearance of misfolded and aggregation-prone proteins increases. The present invention is directed to methods, such as pharmacological methods of achieving a level of cholinergic signaling that restores or maintains proteins homeostasis and decreases the level of misfolded and/or aggregated proteins. In some aspects, the level of cholinergic signaling is sufficient to activate the heat shock response (HSR) in a post-synaptic cell. In another aspect, the level of cholinergic signaling is sufficient to increases the expression of protein chaperones.

As described above, the invention encompasses methods of reducing protein misfolding and/or aggregation in a post-synaptic cell comprising changing the level of cholinergic signaling.

As used herein, “cholinergic hypostimulation-associated protein misfolding and/or aggregation” refers misfolding and/or aggregation of proteins associated with a lower level of cholinergic stimulation that that which would maintain or restore proteostasis and/or activate the heat shock response and/or increase chaperone expression. Cholinergic hypostimulation-associated protein misfolding and/or aggregation can also refer misfolding or aggregation associated with a greater level of GABAergic activity than that which maintain or restore proteostasis.

As used herein, “cholinergic hyperstimulation-associated protein misfolding and/or aggregation” refers misfolding and/or aggregation of proteins associated with a higher level of cholinergic stimulation that that which would maintain or restore proteostasis and/or activate the heat shock response and/or increase chaperone expression. Cholinergic hyperstimulation-associated protein misfolding and/or aggregation can also refer to misfolding or aggregation associated with a lower level of GABAergic activity than that which would maintain or restore proteostasis and/or activate the heat shock response and/or increase chaperone expression.

Cholinergic signaling refers to the activity mediated by the binding of a ligand to a cholinergic receptor. In some aspects, the invention is a method of reducing protein misfolding and/or aggregation in a subject comprising administering an agent that increases cholinergic signaling or an agent that decreases GABAergic signaling. An agent that increases cholinergic signaling can increase cholinergic signaling by directly or indirectly activating a cholinergic receptor. Methods of increasing cholinergic signaling have been described in the literature such as in Shah et al. (2009). Am J Physiol Cell Physiol.; 296(2): C221-C232, the contents of which are expressly incorporated by reference herein. Agents that directly activate the receptor include, for example, cholinergic receptor agonists (also referred to herein as “cholinergic agonists”). Agents that indirectly activate a cholinergic receptor activate the receptor by a mechanism other than binding; such agents include, for example, agents that increase the synthesis and/or release of acetylcholine from the pre-synaptic cell, agents that increase the expression of a cholinergic receptor on the post-synaptic cell, agents that decrease cholinesterase activity (also referred to herein as cholinesterase and as acetylcholinesterase inhibitors), for example, by decreasing expression or increasing degradation, and agents that increase the activity of choline acetyltransferase (ChAT), for example, by increasing expression of the enzyme and/or by decreasing degradation. Cholinergic receptors are membrane proteins that bind acetylcholine. Cholinergic receptors include both the nicotinic and muscarinic receptor families.

A cholinergic agonist is an agonist of a cholinergic receptor. Cholinergic agonists encompass both full and partial agonists. Cholinergic agonists and methods of identifying cholinergic agonists have been described extensively in the literature. Nonlimiting examples of cholinergic agonists include acetylcholine, choline, nicotine, muscarine, carbachol, galantamine, arecoline, cevimeline, levamisole, phenyltrimethylammonium, dimethylphenyl-piperazinium, cytisine, varenciline, epibatidine, oxotremorine, McN-A-343, pilocarpine, bethanechol, cevimeline, and demecarium.

In one aspect, the cholinergic agonist is a nicotinic receptor agonist. In another aspect, the cholinergic agonist is a muscarinic receptor agonist. Nicotinic acetylcholine receptors are a family of ligand-gated, pentameric ion channels and bind acetylcholine and nicotine (Albuquerque et al. (2009), Physiol. Rev. 89(1): 73-120). Muscarinic receptors bind acetylcholine and muscarine and are known to mediate a variety of physiological responses in the central and peripheral nervous systems (Harvey et al. (2009) Pharmacology 4^{th} Edition).

Cholinesterase inhibitors have been described in detail in the art and include, for example, neostigmine, physostigmine, pyridostigmine, rivastigmine, galantamine, donepizil, edrophonium, ambenomium and tacrine. In addition to small molecules, a cholinesterase inhibitor can also be a protein, a peptide, a peptidomimetic, an antibody or a nucleic acid. Nucleic acids include, but are not limited to, DNA, RNA, an RNA interfering agent and PNA.

In one aspect, the cholinergic agonist is an inhibitor of GEI-11 or a homolog thereof. That GEI-11 co-purifies with a subunit of the neuromuscular junction and GEI-11 RNAi confers sensitivity to nicotine has been described in Gottschalk et al. (2005), Embo Journal 24: 2566-78, the contents of which are expressly incorporated by reference herein. In some aspects, the cholinergic agonist is an inhibitor of a mammalian homolog of GEI-11. GEI-11 is, for example, homologous to mammalian SNAPC4 (AceView: a comprehensive cDNA-supported gene and transcripts annotation, Genome Biology 2006, 7 (Suppl 1):S12). Inhibitors of GEI-11 or a homolog can, for example, be a small molecule, a protein, a peptide, a peptidomimetic, an antibody or a nucleic acid. Nucleic acids include, but are not limited to, DNA, RNA, an RNA interfering agent and PNA.

Homologs are defined herein as sequences characterized by nucleotide or amino acid sequence homology. When an equivalent position in the compared sequences is occupied by the same base or amino acid, then the molecules are identical at that position; when the equivalent site occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md.

As described above, the invention also encompasses methods of reducing cholinergic hyperstimulation-associated protein misfolding comprising administering to said subject an effective amount of an agent that decreases cholinergic signaling or an agent that increases GABAergic signaling. An example of an agent that decreases cholinergic signaling is a cholinergic receptor antagonist (also referred to herein as a “cholinergic antagonist”). A cholinergic antagonist is an agent that inhibits a cholinergic receptor, for example, by inhibiting binding of a receptor ligand. An agent that decreases cholinergic signaling also includes, for example, agents that decrease synthesis and/or release of acetylcholine from the pre-synaptic cell, that decrease expression of the cholinergic receptor, that decrease the activity of choline acetyltransferase and/or that increase the activity of a cholinesterase. Cholinergic antagonists and methods of identifying cholinergic agonists have of course been described extensively in the literature. Nonlimiting examples of agents that decrease cholinergic signaling include, for example, scopolamine, mecamylamine, alpha-bungarotoxin, hexamethonium, succinylcholine, tubocurarine, ipratropium, diphenhydramine, triotropium and pharmaceutically acceptable salts thereof. An agent that inhibits the activity of choline acetyltransferase includes, for example, proteins, peptides, peptidomimetics, antibodies or nucleic acids. Nucleic acids include, but are not limited to, DNA, RNA, an RNA interfering agent and PNA.

GABAergic activity refers to activity mediated by gamma amino butyric acid (GABA). Agents that increase GABAergic activity can for example, increase or mimic the effect of GABA. Non-limiting examples of agents that increase GABAergic activity include, for example, gabapentin, gamma-vinyl GABA, valproic acid, progabide, gamma-hydroxybutyric acid, fengabine, cetylGABA, topiramate, tiagabine, acamprosate (homo-calcium-acetyltaurine), and pharmaceutically acceptable salts thereof. Agents that decrease GABA can for example, decrease the effect of GABA and/or inhibit the activity of a GABA receptor. Non-limiting examples of agents that decrease GABAergic activity are bicuculline and metrazol.

The term antibody encompasses monoclonal antibodies, polyclonal antibodies, humanized antibodies, chimeric antibodies, single-chain Fv (scFv), Fab fragment, F(ab′) fragments, intrabodies, and synthetic antibodies.

Antibodies can be raised against an appropriate immunogen, including, for example, GEI-11 or a homolog thereof, acetylcholinesterase or choline acetyltransferase (including synthetic molecules, such as synthetic peptides). Preparation of an immunizing antigen, and polyclonal and monoclonal antibody production can be performed using any suitable technique, including, for example, a phage display. A variety of methods have been described (see e.g., Kohler et al., Nature, 256:495-497 (1975) and Eur. J. Immunol. 6:511-519 (1976); Milstein et al., Nature 266:550-552 (1977); U.S. Pat. No. 4,172,124; Harlow, E. and D. Lane, 1988, Antibodies: A Laboratory Manual, (Cold Spring Harbor Laboratory: Cold Spring Harbor, N.Y.); and Current Protocols In Molecular Biology, Vol. 2 (Supplement 27, Summer '94), Ausubel, F. M. et al., Eds., (John Wiley & Sons: New York, N.Y.), Chapter 11, 1991); the teachings of each of which are incorporated herein by reference). Fragments of antibodies can also be used according to the present invention. Antibodies can be fragmented using conventional techniques and the fragments screened for utility in the same manner as whole antibodies. A Fab fragment of an immunoglobulin molecule is a multimeric protein consisting of the portion of an immunoglobulin molecule containing the immunologically active portions of an immunoglobulin heavy chain and an immunoglobulin light chain covalently coupled together and capable of specifically combining with an antigen. Fab fragments can be prepared by proteolytic digestion of substantially intact immunoglobulin molecules with papain using methods that are well known in the art. However, a Fab fragment may also be prepared by expressing in a suitable host cell the desired portions of immunoglobulin heavy chain and immunoglobulin light chain using any methods known in the art. The antibody can also be an intrabody. An intrabody is an intracellularly expressed antibody, a single-chain antibody molecule designed to specifically bind and inactivate target molecules inside cells. Intrabodies have been used in cell assays and in whole organisms [Chen et al., Hum. Gen. Ther. (1994) 5:595-601; Hassanzadeh et al., Febs Lett. (1998) 16(1, 2):75-80 and 81-86]. Inducible expression vectors can be constructed with intrabodies that react specifically with GEI-11 or a homolog thereof, acetycholinesterase or choline acetyltransferase.

As described above, in some aspects of the invention, the agent that inhibits a target molecule (for example, GEI-11 or a homolog thereof, cholinesterase or choline acetyltransferase) is a nucleic acid. In one embodiment, the nucleic acid is an antisense nucleic acid. The antisense nucleic acid can be RNA, DNA, PNA or any other appropriate nucleic acid molecule. A duplex can form between the antisense sequence and its complementary sense sequence, resulting in inactivation of the gene. The antisense nucleic acid can inhibit gene expression by forming a duplex with an RNA transcribed from the gene, by forming a triplex with duplex DNA, etc. An antisense nucleic acid can be produced, for example, for any gene whose coding sequence is known or can be determined by a number of well-established techniques (e.g., chemical synthesis of an antisense RNA or oligonucleotide (optionally including modified nucleotides and/or linkages that increase resistance to degradation or improve cellular uptake) or in vitro transcription). Antisense nucleic acids and their use are described, for example, in U.S. Pat. Nos. 6,242,258, 6,500,615, 6,498,035, 6,395,544 and 5,563,050, the contents of each of which are herein incorporated by reference.

In another embodiment, the agent that inhibits GEI-11 or a homolog thereof, acetylcholinesterase or choline acetyltransferase, is an RNA interfering agent. An “RNA interfering agent” as used herein, is defined as any agent that interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), and small molecules which interfere with or inhibit expression of a target gene by RNA interference (RNAi). The target gene of the present invention is a gene encoding GEI-11 or a homolog thereof. In another embodiment, the gene encodes agent that inhibits GEI-11 or a homolog thereof. As used herein, “inhibition of target gene expression” includes any decrease in expression or protein activity or level of the target gene or protein encoded by the target gene.

In one embodiment, the RNA interfering agent is a siRNA. The siRNA can be chemically synthesized, can be produced by in vitro transcription, or can be produced within a host cell. In one embodiment, siRNA is a double stranded RNA (dsRNA) molecule of about 15 to about 40 nucleotides in length or about 15 to about 28 nucleotides or about 19 to about 25 nucleotides in length or about 19, 20, 21, or 22 nucleotides in length, and may contain a 3′ and/or 5′ overhang on each strand having a length of about 0, 1, 2, 3, 4, 5, or 6 nucleotides. The length of the overhang is independent between the two strands, i.e., the length of the overhang on one strand is not dependent on the length of the overhang on the second strand.

RNAi also includes small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand can precede the nucleotide loop structure and the antisense strand may follow. These shRNAs can be contained in plasmids, retroviruses, and lentiviruses and expressed from, for example, the pol III U6 promoter, or another promoter (see, e.g., Stewart, et al. (2003) RNA April; 9(4):493-501, incorporated by reference herein).

In addition to RNA, RNA interfering agents can also be comprised of chemically modified nucleotides and non-nucleotides, and also include molecules wherein a ribose sugar molecule is substituted for another sugar molecule or a molecule which performs a similar function. In addition, a non-natural linkage between nucleotide residues may be used, such as a phosphorothioate linkage. The RNA strand can be derivatized with a reactive functional group of a reporter group, such as a fluorophore. Exemplary derivatives are modified at a terminus or termini of an RNA strand, typically the 3′ terminus of the sense strand. Other exemplary derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified, for example, they can be alkylated or halogenated. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can additionally be incorporated.

In another embodiment, the nucleic acid is a ribozyme or a deoxyribozyme. Ribozymes and deoxyribozymes have been shown to catalyze the sequence-specific cleavage of nucleic acid molecules. The cleavage site is determined by complementary pairing of nucleotides in the RNA or DNA enzyme with nucleotides in the target nucleic acid. Thus, RNA and DNA enzymes can be designed to cleave to a nucleic acid molecule, thereby increasing its rate of degradation [Cotten et al, EMBO J. 8: 3861-3866, 1989; Usman et al., Nucl. Acids Mol. Biol. 10: 243, 1996; Usman, et al., Curr. Opin. Struct. Biol. 1: 527, 1996; Sun, et al., Pharmacol. Rev., 52: 325, 2000].

The agent that inhibits GEI-11 or a homolog thereof, cholinesterase or choline acetyltransferase, also encompasses peptide aptamers. Peptide aptamers are peptides or small polypeptides that act as dominant inhibitors of protein function. Peptide aptamers bind specifically to target proteins, blocking their function (Kolonin and Finley, PNAS (1998) 95:14266-14271). Peptide aptamers that bind with high affinity and specificity to GEI-11 or a homolog thereof can be isolated by a variety of techniques known in the art. Peptide aptamers can be isolated from random peptide libraries by yeast two-hybrid screens (Xu et al., PNAS (1997) 94:12473-12478). They can also be isolated from phage libraries (Hoogenboom et al., Immunotechnology (1998) 4:1-20) or chemically generated peptides/libraries.

In some embodiments of the invention, the subject is an animal. Animals include vertebrates and invertebrates, e.g., mammals and non-mammals, including, but not limited to, sheep, dogs, cows, chickens, C. elegans, Drosophila melanogaster, amphibians, reptiles and humans. In one embodiment, the animal is an invertebrate. In another embodiment, the animal is a vertebrate. In a further embodiment, the animal is a mammal. In certain embodiments, the mammal is a human.

The invention also encompasses a method of treating a patient suffering from a condition associated with a dysfunction in protein homeostasis.

A “therapeutically effective amount” or an “effective amount” is an amount which, alone or in combination with one or more other active agents, can control, decrease, inhibit, ameliorate, prevent or otherwise affect one or more symptoms of a disease or condition to be treated and/or achieves a recited effect (for example, a reducing protein misfolding and/or aggregation). An effective amount of the agent to be administered can be determined using methods well-known in the art. One of skill in the art would take into account the mode of administration, the disease or condition (if any) being treated and the characteristics of the subject, such as general health, other diseases, age, sex, genotype, body weight and tolerance to drugs. In the present case, the invention is based on the discovery that changes in cholinergic signaling can affect the level of misfolded and aggregation-prone proteins. Increased levels of misfolded and aggregation-prone proteins, in turn, results in toxicity. The skilled artisan would thus appreciate that an effective amount could, for example, be determined by choosing a starting dose, monitoring the patient for an improvement in symptoms and/or adverse effects and gradually increasing or decreasing the dose until an improvement is observed.

“Treating” or “treatment” includes preventing or delaying the onset of the symptoms, complications, or biochemical indicia of a disease, alleviating or ameliorating the symptoms or arresting or inhibiting further development of the disease, condition, or disorder. A “patient” is a human subject in need of treatment.

The invention encompasses the treatment of a condition associated with a dysfunction in the homeostasis of a protein. Exemplary proteins include glucocerebrosidase, hexosamine A, cystic fibrosis transmembrane conductance regulator, aspartylglucsaminidase, α-galactosidase A, cysteine transporter, acid ceremidase, acid α-L-fucosidase, protective protein, cathepsin A, acid β-glucosidase, acid β-galactosidase, iduronate 2-sulfatase, α-L-iduronidase, galactocerebrosidase, acid α-mannosidase, acid β-mannosidase, arylsulfatase B, arylsulfatase A, N-acetylgalactosamine-6-sulfate sulfatase, acid β-galactosidase, N-acetylglucosamine-1-phosphotransferase, acid sphingmyelinase, NPC-1, acid α-glucosidase, β-hexosamine B, heparin N-sulfatase, α-N-acetylglucosaminidase, α-glucosaminide N-acetyltransferase, N-acetylglucosamine-6-sulfate sulfatase, α-N-acetylgalactosaminidase, α-neuramidase, β-glucuronidase, β-hexosamine A and acid lipase, polyglutamine, α-synuclein, Ab peptide, tau protein, transthyretin, superoxide dismutase, hemoglobin, dystrophin, and poly(A) binding protein 2 (PAPB2).

In one embodiment, the disease associated with a dysfunction in proteostasis is a gain of function disorder. The terms “gain of function disorder,” “gain of function disease,” “gain of toxic function disorder” and “gain of toxic function disease” are used interchangeably herein. A gain of function disorder is a disease characterized by increased aggregation-associated proteotoxicity. In these diseases, aggregation exceeds clearance inside and/or outside of the cell. Gain of function diseases include, but are not limited to neurodegenerative diseases associated with aggregation of polyglutamine, Lewy body diseases, amyotrophic lateral sclerosis, transthyretin-associated aggregation diseases, Alzheimer's disease and prion diseases. Neurodegenerative diseases associated with aggregation of polyglutamine include, but are not limited to, Huntington's disease, dentatorubral and pallidoluysian atrophy, several forms of spino-cerebellar ataxia, and spinal and bulbar muscular atrophy. Alzheimer's disease is characterized by the formation of two types of aggregates: extracellular aggregates of Aβ peptide and intracellular aggregates of the microtubule associated protein tau. Transthyretin-associated aggregation diseases include, for example, senile systemic amyloidoses and familial amyloidotic neuropathy. Lewy body diseases are characterized by an aggregation of α-synuclein protein and include, for example, Parkinson's disease. Prion diseases (also known as transmissible spongiform encephalopathies or TSEs) are characterized by aggregation of prion proteins. Exemplary human prion diseases are Creutzfeldt-Jakob Disease (CJD), Variant Creutzfeldt-Jakob Disease, Gerstmann-Straussler-Scheinker Syndrome, Fatal Familial Insomnia and Kuru.

In a further embodiment, the disease associated with a dysfunction in protein homeostasis is a loss of function disorder. The terms “loss of function disease” and “loss of function disorder” are used interchangeably. Loss of function diseases are a group of diseases characterized by inefficient folding of a protein resulting in excessive degradation of the protein. Loss of function diseases include, for example, cystic fibrosis and lysosomal storage diseases. In cystic fibrosis, the mutated or defective enzyme is the cystic fibrosis transmembrane conductance regulator (CFTR). One of the most common mutations of this protein is ΔF508 which is a deletion (Δ) of three nucleotides resulting in a loss of the amino acid phenylalanine (F) at the 508th (508) position on the protein. Lysosomal storage diseases are a group of diseases characterized by a specific lysosomal enzyme deficiency which may occur in a variety of tissues, resulting in the build-up of molecules normally degraded by the deficient enzyme. The lysosomal enzyme deficiency can be in a lysosomal hydrolase or a protein involved in the lysosomal trafficking Lysosomal storage diseases include, but are not limited to, aspartylglucosaminuria, Fabry's disease, Batten disease, Cystinosis, Farber, Fucosidosis, Galactasidosialidosis, Gaucher's disease (including Types 1, 2 and 3), Gm1 gangliosidosis, Hunter's disease, Hurler-Scheie's disease, Krabbe's disease, a-Mannosidosis, B-Mannosidosis, Maroteaux-Lamy's disease, Metachromatic Leukodystrophy, Morquio A syndrome, Morquio B syndrome, Mucolipidosis II, Mucolipidosis III, Neimann-Pick Disease (including Types A, B and C), Pompe's disease, Sandhoff disease, Sanfilippo syndrome (including Types A, B, C and D), Schindler disease, Schindler-Kanzaki disease, Sialidosis, Sly syndrome, Tay-Sach's disease and Wolman disease.

In another aspect of the invention, the condition to be treated is a muscle wasting condition. A muscle wasting condition is defined herein as one in which muscle wasting, muscle degeneration or muscular dystrophy is a symptom. Muscle wasting is an unintentional loss of weight caused by muscle proteolysis. Muscle wasting conditions include, for example, cachexia (e.g., cancer-related cachexia), age-related muscle wasting, Dejerine Sottas syndrome, starvation, Amyotrophic Lateral Sclerosis (ALS), Spinal Muscular Atrophy Types I, II and III, Spinal Bulbar Muscular Atrophy, dermatomyositis, polymyositis, Inclusion body myosistis, myasthenia gravis, Lambert-Eaton Myasthenic syndrome, Congenital Myasthenic syndromes, Sporadic inclusion body myositis and muscular dystrophies. Muscle wasting also occurs during the aging process (referred to herein as “age-related muscle wasting”). Muscle wasting is additionally associated with catabolic chronic conditions such as diabetes mellitus, renal failure, liver failure, HIV infection, AIDS, and cancer (Castaneda et al., Muscle wasting and protein metabolism, J. Anim. Sci. 80(E. Suppl. 2): E98-E105). Muscle wasting conditions encompass muscular dystrophies which are a group of degenerative diseases associated with progressive skeletal muscle wasting leading to muscle weakness. Exemplary muscular dystrophies are Duchenne muscular dystrophy, Becker muscular dystrophy, Emery-Dreifuss Muscular Dystrophy, Limb-Girdle muscular dystrophy, Fascioscapulohumeral Muscular Dystrophy (also referred to as Landouzy-Dejerine), Myotonic Dystrophy, Oculopharangeal Muscular Dystrophy, Distal Muscular Dystrophy, and Congenital Muscular Dystrophy.

In another embodiment, the invention is a method of treating a patient suffering from a condition associated with a dysfunction in proteostasis comprising administering an agent that increases cholinergic signaling or an agent that decreases GABAergic signaling in combination with the administration of a mechanistically distinct proteostasis regulator. The invention also encompasses a method of treating a patient suffering from a condition associated with a dysfunction in proteostasis comprising administering an agent that decreases cholinergic signaling or an agent that increases GABAergic signaling in combination with the administration of a mechanistically distinct proteostasis regulator or protein chaperone. The term “proteostasis regulator” refers to small molecules, siRNA and biologicals (including, for example, proteins that enhance cellular protein homeostasis) that function by manipulating signaling pathways, including, but not limited to, the heat shock response or the unfolded protein response, or both, resulting in transcription and translation of proteostasis network components. Proteostasis regulators can also regulate protein chaperones by upregulating transcription or translation of the protein chaperone, or inhibiting degradation of the protein chaperone. In addition, proteostasis regulators can upregulate an aggregation pathway or a disaggregase activity. In one aspect, the proteostasis regulator is not a protein chaperone. In yet another aspect, the proteostasis regulator enhances the homeostasis of a mutated protein but does not bind the mutated protein. A mechanistically distinct proteostasis regulator is a proteostasis regulator that enhances cellular proteostasis by a mechanism other than by modulating cholinergic signaling. Exemplary proteostasis regulators are the celastrols, MG-132 and L-type Ca²⁺ channel blockers (e.g., dilitiazem and verapamil). The term “celastrols” refers to celastrol and derivatives or analogs thereof, including, but not limited to, those celastrol derivatives described in Westerheide et al., J Biol Chem, 2004. 279(53): p. 56053-60, the contents of which are expressly incorporated by reference herein. Celastrol derivatives include, for example, celastrol methyl ester, dihydrocelastrol diacetate, celastrol butyl ether, dihydrocelastrol, celastrol benzyl ester, primesterol, primesterol diacetate and triacetate of celastrol.

It is to be understood that an agent is administered in combination with another agent when both agents are part of the same composition and/or when both agents are administered at the same time and/or when both agents are administered at different times.

The form of an agent or pharmaceutical composition comprising said agent used according to the inventive methods of treatment depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the pharmacologic agent or composition. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like. Pharmaceutical compositions can also include large, slowly metabolized macromolecules such as proteins, polysaccharides such as chitosan, polylactic acids, polyglycolic acids and copolymers (such as latex functionalized SEPHAROSE™, agarose, cellulose, and the like), polymeric amino acids, amino acid copolymers, and lipid aggregates (such as oil droplets or liposomes).

For parenteral administration, pharmaceutical compositions or pharmacologic agents can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

The compositions can be prepared as injectable formulations, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The compositions and pharmacologic agents described herein can be administered in the form of a depot injection or implant preparation which can be formulated in such a manner as to permit a sustained or pulsatile release of the active ingredient.

Additional formulations suitable for other modes of administration include oral, intranasal, and pulmonary formulations, suppositories, and transdermal applications.

For suppositories, binders and carriers include, for example, polyalkylene glycols or triglycerides; such suppositories can be formed from mixtures containing the active ingredient in the range of 0.5% to 10%, preferably 1%-2%. Oral formulations include excipients, such as pharmaceutical grades of mannitol, lactose, starch, magnesium stearate, sodium saccharine, cellulose, and magnesium carbonate. Topical application can result in transdermal or intradermal delivery. Transdermal delivery can be achieved using a skin patch or using transferosomes. [Paul et al., Eur. J. Immunol. 25: 3521-24, 1995; Cevc et al., Biochem. Biophys. Acta 1368: 201-15, 1998].

The invention is illustrated by the following non-limiting examples.

### EXEMPLIFICATION

### Example 1

### Cholinergic Imbalance Affects Post-Synaptic Cellular Proteostasis

An imbalance in cholinergic signaling at the neuromuscular junction, by either increased number of functional ACh receptors (increased expression levels and increased sensitivity to cholinergic agonists) or increase in acetylcholine availability, can lead to the activation of stress response, HSR, in the post-synaptic cells. This leads to an increase in chaperone levels, which in turn ameliorate misfolding and aggregation-toxicity phenotypes. Therefore, the “slight” imbalance at the cholinergic/gabaergic level improves post-synaptic proteostasis in disease-like scenarios.

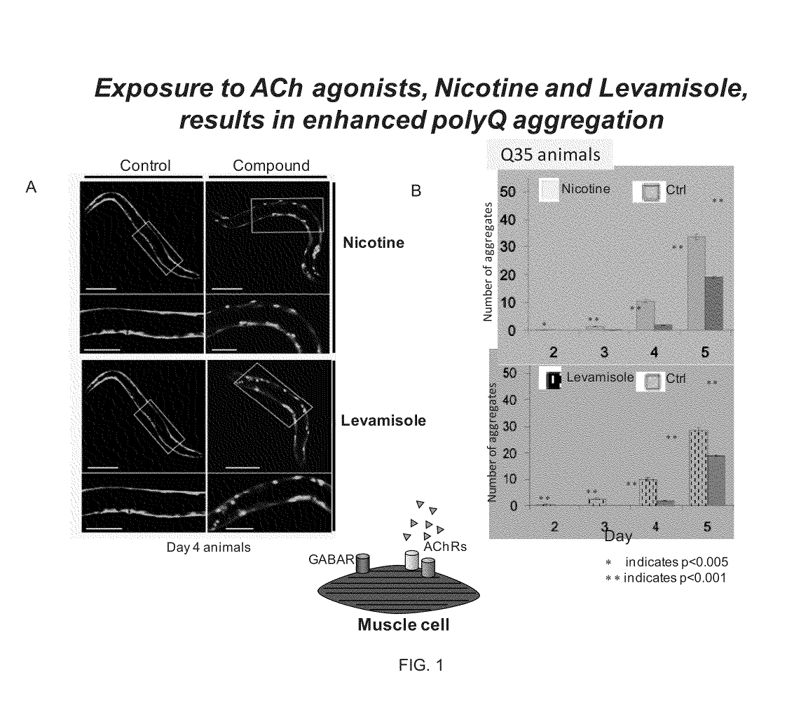

As shown in FIG. 1, treatment of one day old (L1s) Q35 animals with 25 μM levamisole or 1 mM nicotine in a C. elegans model of polyQ aggregation (described, for example, in Garcia et al. (2007), Genes and Development 21: 3006-3016, the contents of which are expressly incorporated by reference) caused increased polyQ aggregation compared to control animals.

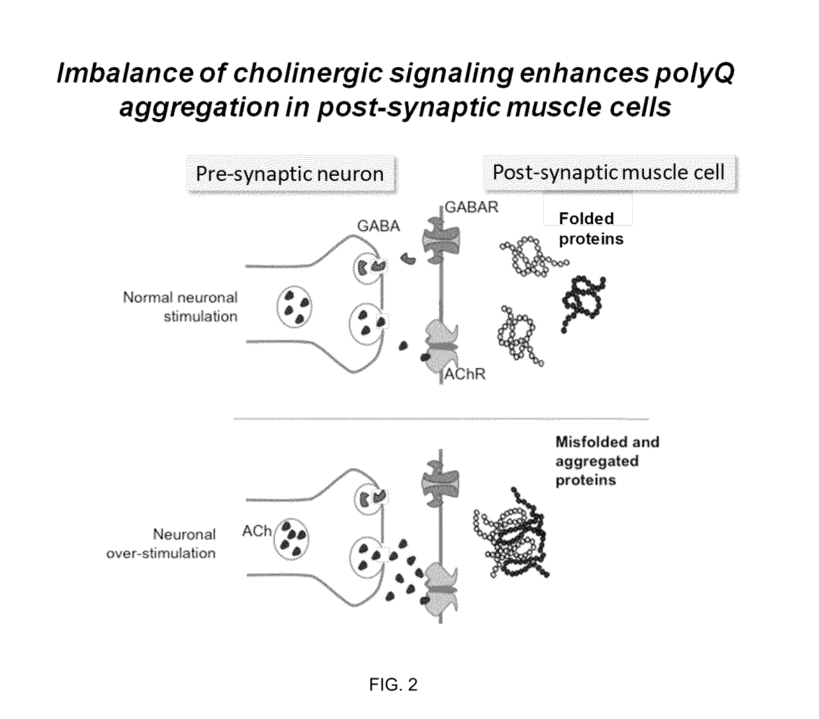

FIG. 2 shows a schematic showing that a strong imbalance in neuronal signaling resulting in overstimulation of post-synaptic cells disrupts the cell's folding capacity and causes misfolding and aggregation of metastable and aggregation-prone proteins (as shown in Garcia et al. 2007).

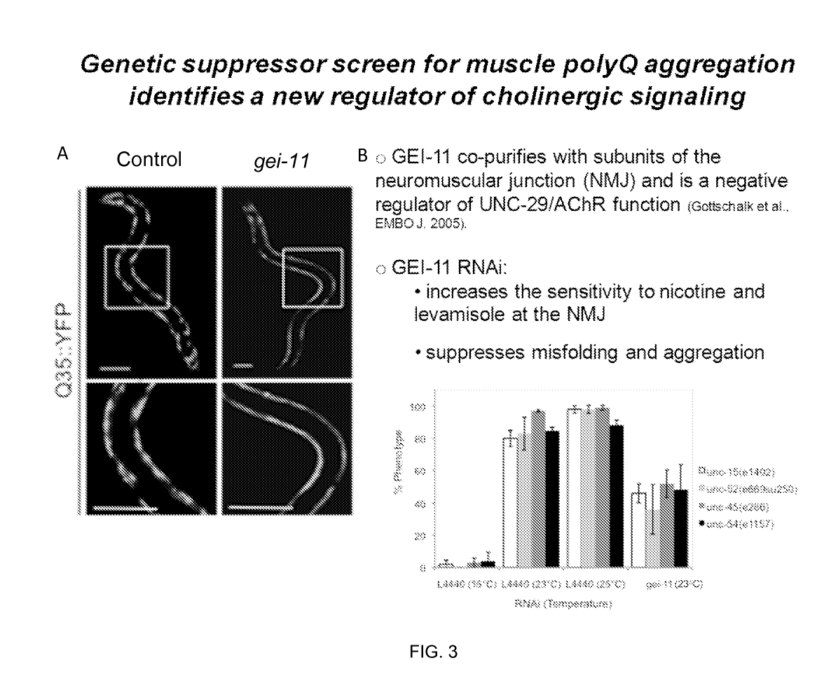

The gei-11 gene was identified from a genome wide screen for suppression of Q35::YFP aggregation in C. elegans muscle cells. As shown in FIG. 3, exposure of animals to gei-11 RNAi bacteria reduced polyQ aggregation as compared to control animals. In addition, the gei-11 gene knockdown was found to also suppress aggregation in a SOD1-G93A model, and to ameliorate the misfolding and toxicity phenotypes associated to temperature sensitive (TS) mutant proteins.

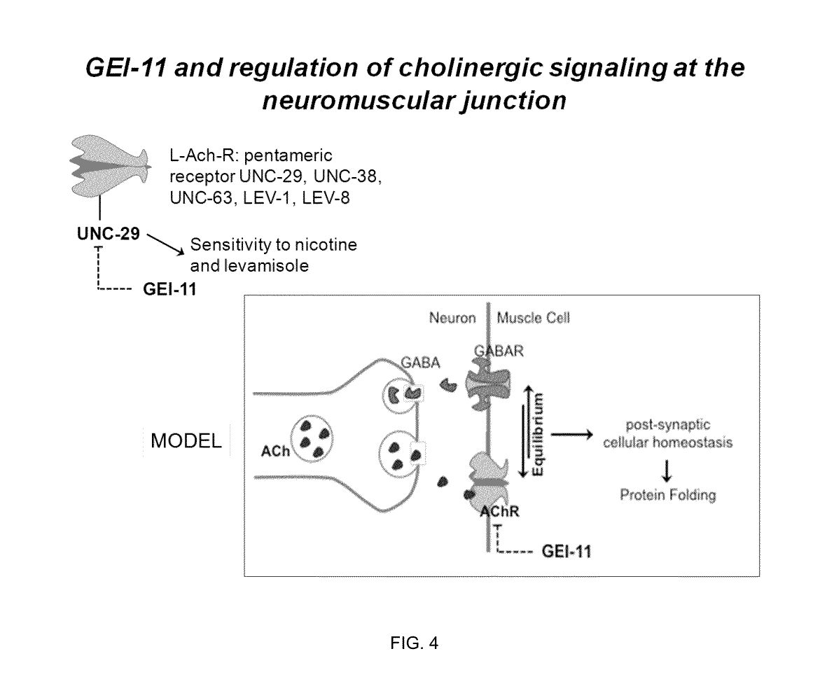

FIG. 4 is a schematic showing the role of GEI-11 in negatively regulating the acetylcholine receptor (function and/or expression).

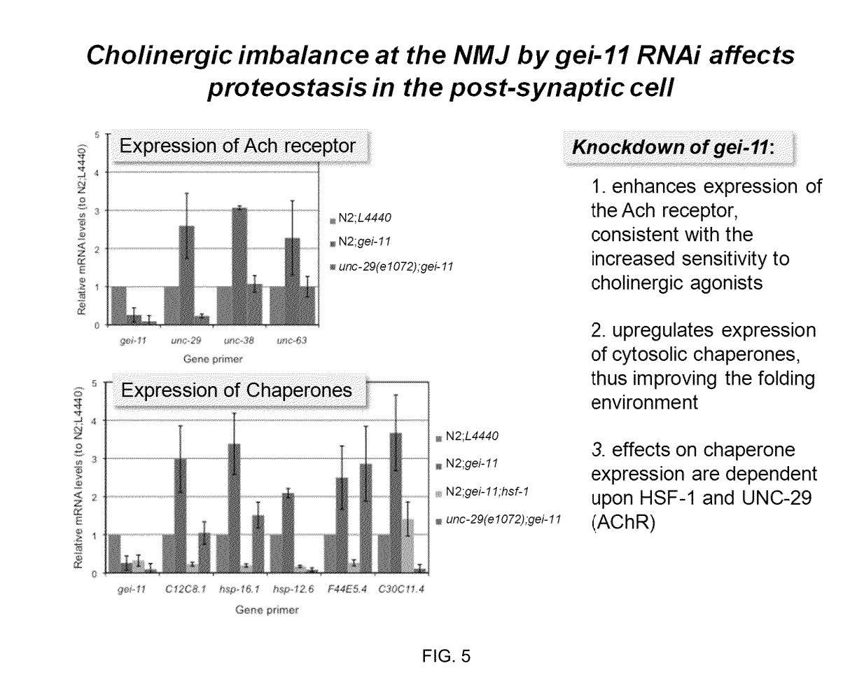

FIG. 5 graphs show relative mRNA levels of the acetycholine receptor (top graph); and chaperones (bottom graph) in N2 animals fed L4440 bacteria (empty vector), N2 animals fed gei-11 RNAi-containing bacteria and an ACh receptor mutant strain (for subunit UNC-29) fed gei-11 RNAi-containing bacteria; for each of the indicated gene primers. These results demonstrate that knockdown of the negative regulator gei-11, leads to an increase in expression of the acetylcholine receptor subunits, and as a consequence HSR is activated (increase in chaperone levels in HSF-1 dependent manner). These results also demonstrate that knockdown of gei-11 upregulates expression of cytosolic chaperones, thus improving the folding environment and that the effects on chaperone expression are dependent on HSF-1 and UNC-29. The UNC-29 mutant strain [unc-29(e1072)] confirms that this effect is fully dependent on the cholinergic activity.

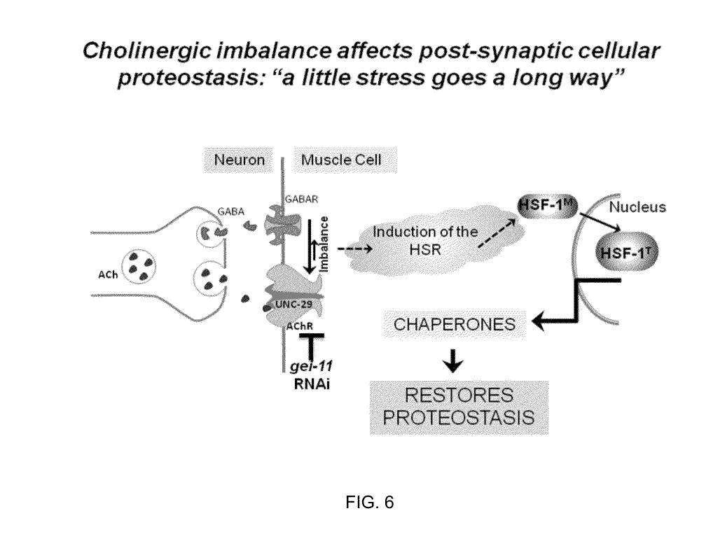

FIG. 6 is a schematic showing that cholinergic imbalance affects post-synaptic cellular proteostasis.

### Materials and Methods

### C. Elegans Strains and Maintenance

Worms were maintained according to standard methods, at 20° C. on nematode growth media (NGM) with OP50 E. coli [S. Brenner, 1974]. The wild-type Bristol strain N2 was obtained from the Caenorhabditis elegans Genetic Center (CGC). The polyglutamine and SOD1 strains have been described elsewhere [J. F. Morley et al., 2002; E. A. Nollen et al., 2004; T. Gidalevitz et al., 2009]. The temperature sensitive strains utilized were: unc-15(e1402), unc-54(e1157), unc-52(e669su250) and unc-45(e286) from CGC(CB1402, CB1157, HE250 and CB286, respectively). The Q35; unc-38(e264) strain was generated by crossing Q35 (AM140) animals with unc-38(e264) (CB904). The mutant unc-29(e1072) was also obtained from CGC (CB1072).

### RNA Interference Screen

The gei-11 gene was identified from a genome wide screen for suppression of Q35::YFP aggregation in C. elegans muscle cells. The screen was performed with the commercial RNAi library (GENESERVICE™, USA) [R. S. Kamath and J. Ahringer, 2003; R. S. Kamath et al., 2003], in a 96-well plate format, by feeding dsRNA-producing bacteria to age-synchronized animals in liquid culture. RNAi bacterial cultures were grown for approximately 8 hours in LB-ampicillin 50 μg/ml (65 μl), at 37° C. with continuous shaking at 315 rpm (Orbital shaker, GENEMACHINES HIGRO®, Genomic Solutions, USA), and induced with 0.5 mM isopropyl β-D-thiogalatoside (IPTG) for 3 hours at 37° C. To obtain an age synchronized population of L1 larvae (first larval state post egg hatching), Q35 gravid adults were bleached with a NaOCl solution [250 mM NaOH and 1:4 (v/v) dilution of commercial bleach] and the eggs were allowed to hatch in M9 buffer overnight at 20° C. 10 to 15 animals in 50 μM9 plus [M9, 10 μg/ml cholesterol, 50 μg/ml ampicillin, 10 μg/ml tetracycline, 0.1 μg/ml fungizone and 170 μg/ml IPTG] were added per well (day 1). The plates were incubated at 20° C. with continuous shaking at 200 rpm (INNOVA™ 4430 Incubator Shaker, New Brunswick, USA) and the animals scored 5 days later (6 days old) for reduction in the number of fluorescent foci using the stereomicroscope Leica MZ16FA equipped for epifluorescence (Leica Microsystems, Switzerland). Suppression of aggregation was scored positive when more than 50% of the worms had a 50% or higher reduction in foci number, without loss of body fluorescence. As a negative control, animals were fed bacteria carrying the L4440 empty vector.

gei-11 RNAi Effect in Other Misfolding Models

The gei-11 gene knockdown was found to also suppress aggregation in a SOD1-G93A model, and to ameliorate the misfolding and toxicity phenotypes associated to temperature sensitive (TS) mutant proteins.

SOD1-G93A aggregation suppression was scored in a similar way to the polyQ. NGM plates supplemented with 50 μg/ml ampicillin, 1 mM IPTG and 6 μg/ml tetracycline were seeded with overnight RNAi bacteria culture, and induced with IPTG (0.5 mM) for 3 hours. SOD1 age synchronized L1 animals were transferred onto these plates, grown at 20° C. for 4 days and scored for a reduction in the number of fluorescent foci.

For the TS strains, animals were age-synchronized to L1 stage, grown on gei-11 RNAi plates at a sensitized temperature of 23° C. (the permissive temperature being 15° C. and the restrictive temperature 25° C.), and scored for each specific phenotype 4 days later (5 days old). For abnormal body shape (or stiff paralysis in the unc-52(e669su250)), partially paralyzed animals with moving heads and stick-like bodies were scored. For the egg laying defect in the unc-45(e286) animals were scored for accumulation of eggs in the gonad and enlargement of the animal belly. For the slow moving animals (unc-15(e1402), unc-54(e1157)), 15-20 animals were placed in a freshly seeded plate equilibrated to 20° C. and those that were completely paralyzed were scored. As a control experiment, L1 nematodes were grown on control bacteria (L4440 vector RNAi) at either 15° C., 23° C. or at 15° C. until L3-L4 stage (to avoid embryonic and developmental phenotypes), transferred to 25° C. and scored 2 days later for the same phenotype. Experiments were repeated until the n value was at least 100.

### Drug Assay for Levamisole and Nicotine Sensitivity

NGM plates supplemented with 50 μg/ml ampicillin, 1 mM IPTG and 6 μg/ml tetracycline were seeded with overnight RNAi bacteria cultures (gei-11 or L4440 vector control), induced with IPTG (0.5 mM) for 3 hours. Q35 age synchronized L1 animals were transferred onto these plates and grown at 20° C. for 4-5 days. At each time point, 25-30 animals were transferred to freshly prepared NGM plates containing 1 mM Levamisole (Sigma), 30 mM of Nicotine (Sigma) or the solvent (water or ethanol) control. Sensitivity to each of the cholinergic agonists was followed by visual inspection every 2 to 5 min and defined as paralysis, or lack of movement in response to prodding on the nose or tail of the animal. The experiment was done in triplicate, with an n value of at least 60 for each one. Q35; unc-38(e264) and unc-29(e1072) mutants strains were used to correlate aggregation suppression and specificity to cholinergic receptors' function. Compound preparation: stock solution of 800 mM levamisole (Sigma) in water; stock solution of 300 mM nicotine (Sigma) prepared in ethanol.

### Acetylcholine and GABA Titration Assay

This assay was performed in 96-well plates in liquid culture (C. Voisine et al. 2007). Each well contained a final volume of 60 μl comprising 15-20 L1 animals, drugs at the appropriate concentration and OP50 bacteria to a final OD_{595 nm} of 1.2 in the microtiter plate. Animals and bacteria were resuspended in S-media complete supplemented with streptomycin, penicillin and nystatin (Sigma), and grown with continuous shaking at 200 rpm at 20° C. (INNOVA™ 4430 Incubator Shaker, New Brunswick, USA) for 5 days. At this time animals were transferred from the 96-well microtiter liquid cultures onto plates and the total number of aggregates was scored for each individual (viewed at 100× magnification with a stereomicroscope equipped for epifluorescence). Compound preparation: stock solutions of 500 mM GABA (Sigma) and 550 mM Acetylcholine (Sigma) were prepared in water.

### Quantitative Real-Time PCR

Supplemented NGM plates were seeded with overnight RNAi bacteria cultures, induced with IPTG (0.5 mM) for 3 hours. Age synchronized L1 animals were transferred onto the plates and grown at 20° C. for 5 days. Animals were collected and washed with M9 buffer, pelleted at 3,000 g (Eppendorf centrifuge 5424, Eppendorf) and resuspended in Trizol (Invitrogen #15596-026). Samples were homogenized by vortexing for 10 min, followed by 3 cycles of freeze (liquid nitrogen)-thaw (37° C.). 100 μl of chloroform were added to each sample, followed by vortexing and centrifugation at 13,500 g for 15 min (4° C.). A volume of 300 μl of 2-Propanol was added to each aqueous layer (10 min incubation) and total RNA was spun down at 18,000 g (4° C.) for 10 min. The pellets were washed with 75% (v/v) ethanol, air-dried and resuspended in nuclease free water at 60° C. for 10 min. RNA aliquots of 10 μg were used for DNase treatment (Applied Biosystems #AM1906). 1 μg of purified RNA was used for cDNA synthesis (Bio-Rad #170-8891). cDNA samples were diluted to a final volume of 500 μl in water and 8 ng were used for real-time PCR amplification in 25 μl reaction volumes, with specific primers for each gene. Quantitative PCR was performed using IQ™ SYBR® Green Supermix (Bio-Rad, Catalog #170-8880) in the iCycler system (Bio-Rad), in thin wall 200 μl PCR plates (Cat. No. 223-9441) sealed with the optical quality sealing tape (Cat. No. 223-9444). The relative amounts of gene-mRNA were determined using the Comparative C_{T} Method for quantitation [Real-Time PCR Applications Guide, Bio-Rad]. mRNA levels within an experiment were normalized relative to actin mRNA as the internal control, and then determined relative to non-RNAi treated samples (L4440 vector control). C_{T} values were obtained in triplicate for each sample (technical triplicate), and the experiment was repeated at least three times. Forward and reverse primers were used in this experiment for measuring gei-11, unc-29, unc-38, unc-63, CI2C8.1, C30C11.4, F44E5.4, hsp-16, hsp-12, hsf-1, and act-1. unc-29(e1072) was used to test dependence on cholinergic receptors function, and hsf-1 RNAi was used to test HSF-1 dependent up-regulation of chaperones.

Additional methods are described in Garcia et al. (2007), Genes and Development 21: 3006-16.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of reducing protein misfolding and/or aggregation in a subject comprising administering to said subject an effective amount of an agent that increases cholinergic signaling or an agent that decreases GABAergic activity.

2. The method of claim 1, wherein an agent that increases cholinergic signaling is administered.

3. The method of claim 1, wherein the agent that increases cholinergic signaling is a cholinergic agonist.

4. The method of claim 3, wherein the cholinergic agonist is a nicotinic receptor agonist.

5. The method of claim 3, wherein the cholinergic agonist is a muscarinic receptor agonist.

6. The method of claim 3, wherein the cholinergic agonist is selected from the group consisting of acetylcholine, choline, nicotine, muscarine, carbachol, galantamine, arecoline, cevimeline, levamisole, phenyltrimethylammonium, dimethylphenyl-piperazinium, cytosine, epibatidine, oxotremorine, McN-A-343, pilocarpine, bethanechol, cevimeline and demecarium.

7. The method of claim 2, wherein the agent that increases cholinergic signaling is a cholinesterase inhibitor.

8. The method of claim 7, wherein the cholinesterase inhibitor is selected from the group consisting of neostigmine, physostigmine, pyridostigmine, rivastigmine, galantamine, donepizil, edrophonium, ambenomium and tacrine.

9. The method of claim 4, wherein the agent that increases cholinergic signaling inhibits the activity of GEI-11 or a homolog thereof.

10. The method of claim 9, wherein the agent inhibits the activity of a mammalian homolog of GEI-11.

11. A method of treating a condition mediated by protein dysfunction in a patient in need thereof comprising administering to said patient an effective amount of an agent that increases cholinergic signaling.

12. The method of claim 11, wherein the condition is a loss of function disorder.

13. The method of claim 11, wherein the condition is a gain of function disorder.

14. The method of claim 11, wherein the condition is a muscle wasting condition.

15. The method of claim 14, wherein the muscle wasting condition is selected from the group consisting of cachexia, age-related muscle wasting, Dejerine Sottas syndrome, starvation, Amyotrophic Lateral Sclerosis (ALS), Spinal Muscular Atrophy Types I, II and III, Spinal Bulbar Muscular Atrophy, dermatomyositis, polymyositis, Inclusion body myosistis, myasthenia gravis, Lambert-Eaton Myasthenic syndrome, Congenital Myasthenic syndromes and muscular dystrophies.

16. A method of reducing cholinergic hyperstimulation-associated protein misfolding, aggregation or both in a subject comprising administering to said subject an effective amount of an agent that decreases cholinergic signaling or an agent that increases GABAergic activity.

17. The method of claim 16, wherein the agent that decreases cholinergic signaling is a cholinergic antagonist or a cholinesterase activator.

18. A method of reducing protein misfolding and/or aggregation in a post-synaptic muscle cell in a subject in need thereof comprising increasing cholinergic signaling at the neuromuscular junction.

19. A method of reducing protein misfolding, aggregation or both in a post-synaptic muscle cell comprising decreasing cholinergic signaling at the neuromuscular junction.

20. A method of screening for an agent that reduces protein misfolding, aggregation or both in a post-synaptic cell comprising screening a candidate agent for cholinergic agonist activity.
